(19) United States
(12) Patent Application Publication
(10) Pub. No.: US 2026/0216207 A1
(43) Pub. Date: Jul. 30, 2026

(54) 7-CARBONYLSTAUROSPORINE DERIVATIVE, PREPARATION METHOD THEREFOR, AND USE THEREOF

(71) Applicant: HANGZHOU KEXING BIOCHEM. CO., LTD., Hangzhou, ZHEJIANG PROVINCE (CN)

(72) Inventors: ZHONGJUN MA, ZHOUSHAN, ZHEJIANG PROVINCE (CN); JINHUI WANG, ZHOUSHAN, ZHEJIANG PROVINCE (CN); WANJING DING, ZHOUSHAN, ZHEJIANG PROVINCE (CN); YIJUN MA, ZHOUSHAN, ZHEJIANG PROVINCE (CN); MEIXING LIU, ZHOUSHAN, ZHEJIANG PROVINCE (CN)

(21) Appl. No.: 19/151,986

(22) PCT Filed: Jan. 3, 2024
(86) PCT No.: PCT/CN2024/070274

§ 371 Date: Jul. 29, 2025

(30) Foreign Application Priority Data
Jan. 29, 2023 (CN) 202310043651.8

(51) Int. Cl. A61K 31/553; C07D 498/22

(52) U.S. Cl. CPC A61K 31/553; C07D 498/22

(57) ABSTRACT

The present invention discloses a 7-carbonylstaurosporine derivative, a preparation method therefor, and use thereof. The present invention further discloses a chemical synthesis method for 18 typical compounds of this type, which has a simple synthetic route, is easy to operate and implement, and has easily purchasable required reagents. The compounds disclosed in the present invention have significant inhibitory activity against a PIM-1 kinase and excellent kinase selectivity, and can be used for development of therapeutic drugs for related diseases or disorders caused by abnormal expression of the PIM-1 kinase.

### FIELD OF TECHNOLOGY

The present invention relates to the technical field of medicine, and particularly relates to a 7-carbonylstaurosporine derivative, a preparation method therefor, and use thereof in the field of medicine. The 7-carbonylstaurosporine derivative of the present invention can be used as or for preparing an agent with low side effects for selectively inhibiting PIM-1, and is particularly applicable to treatment of diseases or disorders associated with PIM-1 activity.

### BACKGROUND TECHNOLOGY

PIM kinases belong to a calcium/calmodulin kinase (CAMK) family, and mainly have three subtypes including PIM-1, PIM-2, and PIM-3. Their amino acid sequences have high homology, the PIM-1 and the PIM-2 have a similarity of 61%, the PIM-1 and the PIM-3 have a similarity of 71%, and the PIM-2 and the PIM-3 have a similarity of 44%.

The three subtypes of the PIM kinases have different expression amounts in different tissues. The PIM-1 is highly expressed in hematopoietic cells, lymphocytes, and prostate cells; the PIM-2 is highly expressed in the lymphocytes and brain cells; and the PIM-3 is highly expressed in mammary gland cells, nephrocytes, and the brain cells.

Overexpression of the PIM kinases not only leads to proliferation of cancer cells, but also leads to drug resistance of the cancer cells to conventional therapeutic strategies such as chemotherapy, radiotherapy, and immunosuppressants such as rapamycin. Interactions between the PIM kinases and other oncogenic pathways lead to growth, proliferation, and evading apoptosis of the cancer cells, such as PI3K/mTOR/AKT signaling pathways. A variety of cancer-promoting signaling molecules are downstream targets of the PIM kinases, such as MYC, CDC25, BAD, etc., while expression of the PIM kinases is regulated by pathways such as JAK-STAT and NF-1B.

Since the PIM-2 and the PIM-3 have relatively scarce structural information, the PIM-1 has become a most extensively studied member of a PIM kinase family. According to more than twenty years of development and research, numerous PIM-1 kinase inhibitors with different heterocyclic scaffolds have been designed, but only few compounds have entered clinical studies, and no PIM-1 kinase inhibitors have been approved for marketing currently.

Staurosporine is a broad-spectrum kinase inhibitor and is a most representative indolocarbazole compound, and its several derivatives, such as Midostaurin, Lestaurtinib, and UCN-01, have been approved for marketing or have entered clinical studies, indicating that the compounds have great development prospects. However, poor kinase selectivity and great side effects have always been important factors hindering further development of the compounds.

Through research and summarization of structure-activity relationships of inhibitory effects of existing indolocarbazole compounds on the PIM-1 kinase, the inventor has found that carbonyl at site C-7 is extremely important for inhibiting activity of the PIM-1 kinase. Therefore, 7-carbonylstaurosporine is subjected to structural modification to obtain derivatives with highly selective inhibitory activity against the PIM-1 kinase.

In the present invention, the 7-carbonylstaurosporine as a lead compound is subjected to chemical synthesis to obtain a series of derivatives, and inhibitory effects of this type of compounds on the PIM-1 kinase are determined. Results show that the 7-carbonylstaurosporine derivatives have good inhibitory effects on the PIM-1, and particularly, compound 17 has a significant inhibitory effect on the PIM-1 kinase and has an IC₅₀ of 5.9 nM, thus having a superior effect than a positive control drug TP-3654 (with an IC₅₀ of 31.1 nM). In addition, it is found through further investigation that the compound 17 has an excellent selective inhibitory effect on the PIM-1 kinase, thus having a great development prospect.

The present invention can provide a reference for development of drugs for treating diseases or disorders caused by abnormal expression of the PIM-1 kinase using this type of compounds.

### SUMMARY OF THE INVENTION

The present invention provides a 7-carbonylstaurosporine derivative or a pharmaceutically acceptable salt thereof, where the 7-carbonylstaurosporine derivative has a structure of formula (I) below:

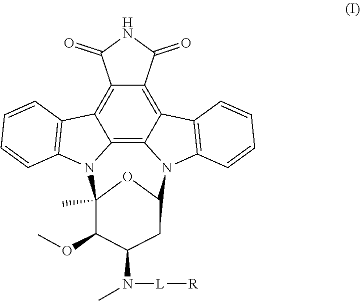

in the formula (I):
L is selected from —(CH₂)ₙ—, —(CH₂)ₙNH—, —(CH₂)ₙO—, —(CH₂)ₙC(═O)O—, —(CH₂)ₙC(═O)NH—, —(CH₂)ₙS—, —(CH₂)ₙS(═O)—, —(CH₂)ₙS(═O)₂—, —(OCH₂CH₂O)ₙ—, —(CH₂CH₂O)ₙ—, —(OCH₂CH₂OCH₂)ₙ—, —(CH₂CH₂OCH₂)ₙ—, —(CH₂CH₂OCH₂CH₂)ₙ—, alkenylene, alkynylene, cycloalkylene, heteroaromatic hydrocarbylene, or any combination thereof, where n represents a natural number from 1 to 10;
R is selected from H, halogen, hydroxy, amino, cyano, nitro, C₁₋₆ alkyl, C₂₋₆ alkenyl, C₂₋₆ alkynyl, C₃₋₁₀ cyclohydrocarbyl, 3-10 membered heterocyclyl, —C(═O)R₁, —OC(═O)R₁, —C(═O)OR₁, —OR₁, —SR₁, —S(═O)R₁, —S(═O)₂R₁, —S(═O)₂NR₁R₂, —NR₁R₂, —C(═O)NR₁R₂, —NR₁C(═O)R₂, —NR₁C(═O)OR₂, —NR₁S(═O)₂R₂, —NR₁C(═O)NR₁R₂, —C₁₋₆ alkylene-NR₁R₂, —C₁₋₆ alkylene-O(P═O)(OH)₂, and —O—C₁₋₆ alkylene-NR₁R₂;
the above alkyl, alkylene, alkenyl, alkynyl, cyclohydrocarbyl, and heterocyclyl at each occurrence are each optionally substituted with one or more substituents independently selected from the following: halogen, hydroxy, oxo, amino, cyano, nitro, C₁₋₆ alkyl, C₂₋₆ alkenyl, C₂₋₆ alkynyl, C₃₋₆ cyclohydrocarbyl, 3-10 membered heterocyclyl, ═N—OR₁, —C(═NH)NH₂, —C(═O)R₁, —OC(═O)R₁, —C(═O)OR₁, —OR₁, —SR₁, —S(═O)R₁, —S(═O)₂R₁, —S(═O)₂NR₁R₂, —NR₁R₂, —C(═O)NR₁R₂, —NR₁C(═O)R₂, —NR₁C(═O)OR₂, —NR₁S(═O)₂R₂, —NR₁C(═O)NR₁R₂, —C₁₋₆ alkylene-NR₁R₂, and —O—C₁₋₆ alkylene-NR₁R₂;
R₁ and R₂ at each occurrence are each independently selected from H, C₁₋₆ alkyl, C₃₋₁₀ cyclohydrocarbyl, and 3-10 membered heterocyclyl; or when R₁ and R₂ are connected to a same nitrogen atom, R₁ and R₂ optionally form a 3-12 membered heterocycle together with the atom to which they are connected.

Further, the 7-carbonylstaurosporine derivative may be selected from any one of the following compounds 1-18:

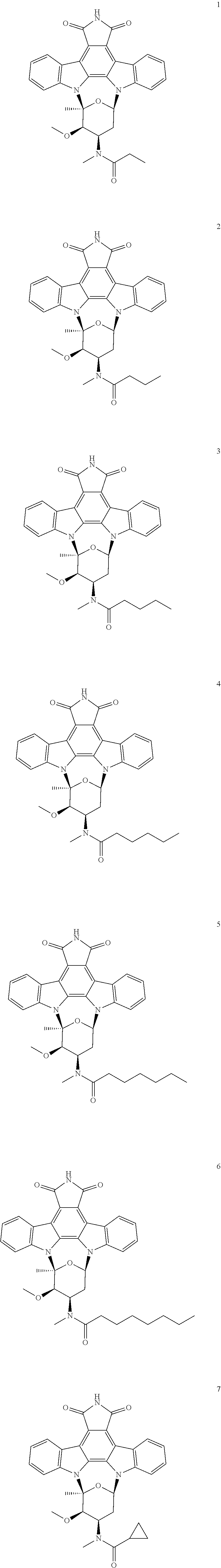

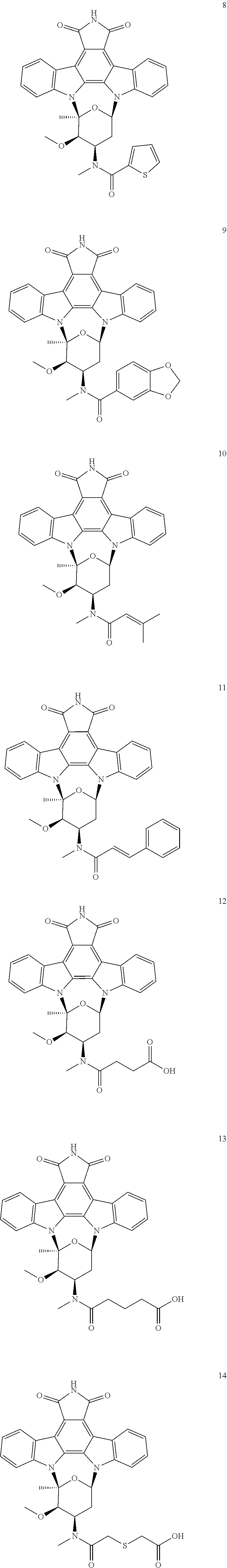

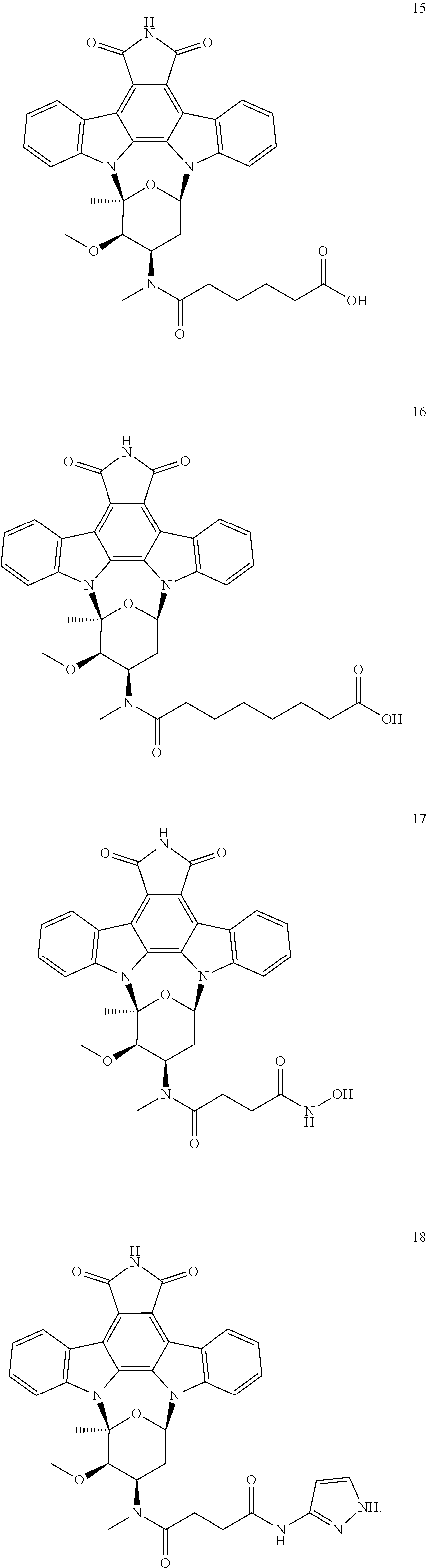 [SMILES: CO[C@@H]1[C@H](N(C)C(=O)CCCCC(=O)O)C[C@H]2O[C@]1(C)n1c3ccccc3c3c4c(c5c6ccccc6n2c5c31)C(=O)NC4=O ; CO[C@@H]1[C@H](N(C)C(=O)CCCCCCC(=O)O)C[C@H]2O[C@]1(C)n1c3ccccc3c3c4c(c5c6ccccc6n2c5c31)C(=O)NC4=O ; CO[C@@H]1[C@H](N(C)C(=O)CCC(=O)NO)C[C@H]2O[C@]1(C)n1c3ccccc3c3c4c(c5c6ccccc6n2c5c31)C(=O)NC4=O ; CO[C@@H]1[C@H](N(C)C(=O)CCC(=O)Nc2cc[nH]n2)C[C@H]2O[C@]1(C)n1c3ccccc3c3c4c(c5c6ccccc6n2c5c31)C(=O)NC4=O]

The present invention further provides a pharmaceutical composition, including the 7-carbonylstaurosporine derivative or a pharmaceutically acceptable salt thereof, and one or more pharmaceutically acceptable carriers, diluents, or excipients.

The present invention further provides use of the 7-carbonylstaurosporine derivative or a pharmaceutically acceptable salt thereof or the pharmaceutical composition in preparation of a drug for inhibiting a PIM-1 kinase.

The present invention further provides use of the 7-carbonylstaurosporine derivative or a pharmaceutically acceptable salt thereof or the pharmaceutical composition in preparation of a drug for treating and/or preventing a disease associated with abnormal activity of a PIM-1 kinase.

The disease may be cancer.

The cancer is preferably leukemia, lymphoma, multiple myeloma, breast cancer, endometrial cancer, ovarian cancer, vaginal cancer, carcinoma of fallopian tube, cervical cancer, renal cancer, bladder cancer, urothelial carcinoma, urethral carcinoma, prostate cancer, testicular cancer, colorectal cancer, sarcoma, bone cancer, myxoma, rhabdomyoma, leiomyoma, fibroma, lipoma, teratoma, pharyngolaryngeal cancer, nasopharyngeal carcinoma, oral cancer, lung cancer, alveolar carcinoma, mesothelioma, carcinoma of small intestine, gastric cancer, esophageal cancer, pancreatic cancer, liver cancer, cholangiocarcinoma, neurofibroma, neuroglioma, neuroblastoma, neuroblastoma, melanoma, skin cancer, basal cell carcinoma, squamous cell carcinoma, thyroid cancer, head and neck cancer, salivary gland cancer, or gastrointestinal stromal tumor.

Preferably, the lymphoma is Hodgkin's disease or non-Hodgkin's lymphoma (e.g., mantle cell lymphoma, diffuse large B-cell lymphoma, follicular center lymphoma, marginal zone B-cell lymphoma, lymphoplasmacytic lymphoma, and peripheral T-cell lymphoma); the lung cancer is non-small cell lung cancer (NSCLC) (including squamous cell carcinoma, adenocarcinoma, and large cell carcinoma, etc.) or small cell lung cancer (SCLC); the renal cancer is renal cell carcinoma, clear cell carcinoma, and renal oncocytoma; the leukemia is chronic lymphocytic leukemia (CLL), acute lymphoblastic leukemia (ALL), T-cell acute lymphoblastic leukemia (T-ALL), chronic myelogenous leukemia (CML), or acute myeloid leukemia (AML); the colorectal cancer is colon cancer or rectal cancer; and the sarcoma is chondrosarcoma.

The present invention further relates to a method for inhibiting a PIM-1 kinase, which includes administering to a subject in need a therapeutically effective amount of the 7-carbonylstaurosporine derivative or a pharmaceutically acceptable salt thereof or the pharmaceutical composition including the same.

The present invention further relates to a method for treating and/or preventing a disease associated with abnormal activity of PIM-1, which includes administering to a subject in need a therapeutically effective amount of the 7-carbonylstaurosporine derivative or a pharmaceutically acceptable salt thereof or the pharmaceutical composition including the same.

Covering contents of the disease may be the same as described above.

The active compound may be formulated in a form suitable for administration by any appropriate route, and the composition of the present invention may be formulated by a conventional method using one or more pharmaceutically acceptable carriers. Therefore, the active compound of the present invention may be formulated into various dosage forms for oral administration, injection (e.g., intravenous, intramuscular, or subcutaneous) administration, and inhalation or insufflation administration. The compound of the present invention may also be formulated into dosage forms such as a tablet, a hard or soft capsule, an aqueous or oily suspension, an emulsion, an injection solution, a dispersible powder or granule, a suppository, a lozenge, or a syrup.

As general guidance, the active compound is preferably in the form of a unit dose, or in the form of a single dose suitable for self-administration by a patient. The unit dose of the compound or composition of the present invention may be expressed in the form of a tablet, a capsule, a sachet, a bottled medicine liquid, a medicine powder, a granule, a lozenge, a suppository, a regenerated medicine powder, or a liquid preparation. The suitable unit dose may be 0.1-1,000 mg.

The pharmaceutical composition of the present invention may include, in addition to the active compound, one or more auxiliary materials, and the auxiliary materials are selected from the following ingredients: a filler (diluent), an adhesive, a wetting agent, a disintegrant, or an excipient, etc. According to different administration methods, the composition may include 0.1-99% by weight of the active compound.

A tablet includes an active ingredient and a non-toxic pharmaceutically acceptable excipient suitable for tablet preparation for mixing. The excipient may be an inert excipient, a granulating agent, a disintegrant, an adhesive, or a lubricant. The tablet may be uncoated or may be coated by known techniques to mask the taste of a drug or to delay disintegration and absorption in the gastrointestinal tract to provide a sustained release effect over a longer period of time.

An oral preparation may also be provided in a soft gelatin capsule in which an active ingredient is mixed with an inert solid diluent or the active ingredient is mixed with a water-soluble carrier or an oil vehicle.

An aqueous suspension includes an active substance and an excipient suitable for aqueous suspension preparation for mixing. The excipient is a suspending agent, a dispersing agent, or a wetting agent. The aqueous suspension may also include one or more preservatives, one or more colorants, one or more flavoring agents, and one or more sweeteners.

An oily suspension may be formulated by suspending an active ingredient in vegetable oil or mineral oil. The oily suspension may include a thickener. The aforementioned sweeteners and flavoring agents may be added to provide a palatable preparation. These compositions may be protected by adding an antioxidant.

The pharmaceutical composition of the present invention may also be in the form of an oil-in-water emulsion. An oil phase may be vegetable oil, mineral oil, or a mixture thereof. A suitable emulsifier may be a naturally occurring phospholipid, and the emulsion may also include a sweetener, a flavoring agent, a preservative, and an antioxidant. Such preparation may also include a buffering agent, a preservative, a colorant, and an antioxidant.

The pharmaceutical composition of the present invention may be in the form of a sterile injectable aqueous solution. An acceptable vehicle or solvent that may be used includes water, a Ringer's solution, and an isotonic sodium chloride solution. A sterile injectable preparation may be a sterile injectable oil-in-water microemulsion in which an active ingredient is dissolved in an oil phase, and an injection solution or the microemulsion may be injected into a bloodstream of a patient by local massive injection. Alternatively, the solution and the microemulsion are preferably administered in a manner that can maintain a constant circulating concentration of the compound of the present invention. To maintain such constant concentration, a continuous intravenous drug delivery device may be used. An example of such device is a Deltec CADD-PLUS™5400 intravenous injection pump.

The pharmaceutical composition disclosed in the present invention may be in the form of a sterile injectable aqueous or oily suspension for intramuscular and subcutaneous administration.

The suspension may be formulated by known techniques using the suitable dispersing agent or the wetting agent and the suspending agent as described above. A sterile injectable preparation may also be a sterile injectable solution or suspension prepared in a parenterally acceptable non-toxic diluent or solvent. In addition, sterile fixed oil may conveniently be used as a solvent or a suspending medium. For this purpose, any blended fixed oil may be used. In addition, fatty acids may also be used for preparing injections.

The compound of the present invention may be administered in the form of a suppository for rectal administration. These pharmaceutical compositions may be prepared by mixing a drug with suitable non-irritating excipients that are solids at ordinary temperature but liquids in the rectum and can be melted in the rectum to release the drug.

The compound of the present invention may be administered as a water-suspensible dispersible powder or granule by adding water. These pharmaceutical compositions may be prepared by mixing an active ingredient with a dispersing agent or a wetting agent, a suspending agent, or one or more preservatives.

As is well known to those skilled in the art, an administration dose of a drug depends on various factors, including but not limited to the following factors: activity of a specific compound used, severity of a disease, age of a patient, weight of the patient, health conditions of the patient, behaviors of the patient, diets of the patient, an administration time, an administration mode, an excretion rate, drug combinations, etc.; and in addition, an optimal therapy method, such as a treatment mode, a daily dose, or a type of a pharmaceutically acceptable salt, may be verified according to a conventional therapeutic regimen.

The present invention further provides a method for preparing compounds 1-18, which includes the following synthetic route with 7-carbonylstaurosporine as an initial substrate:

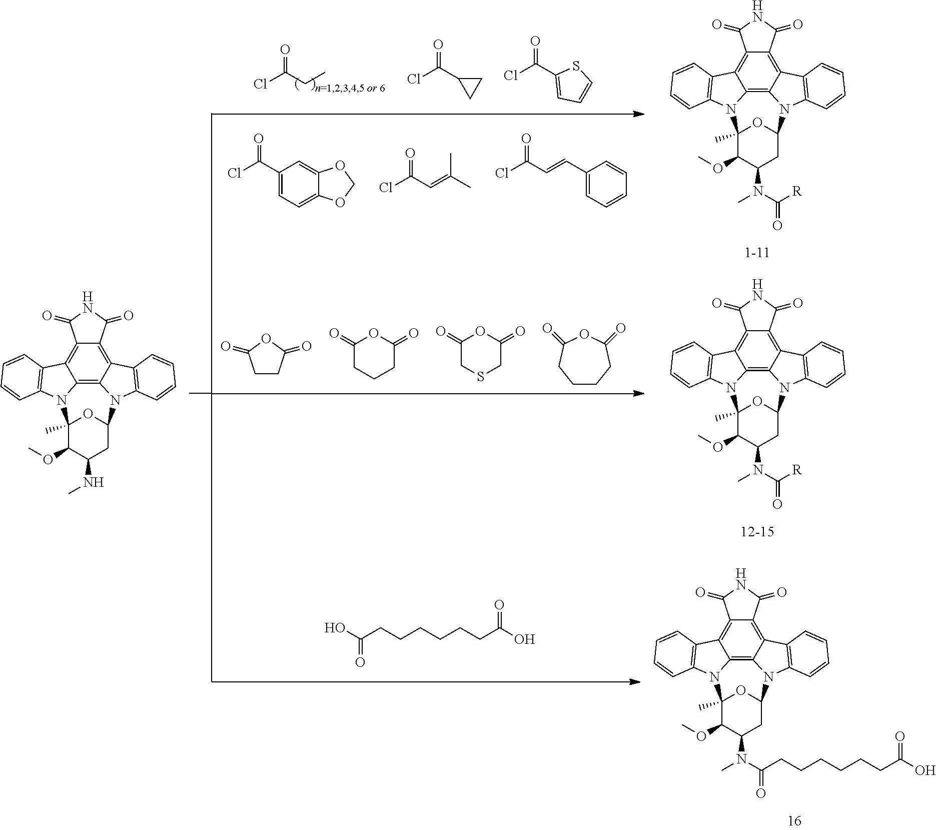

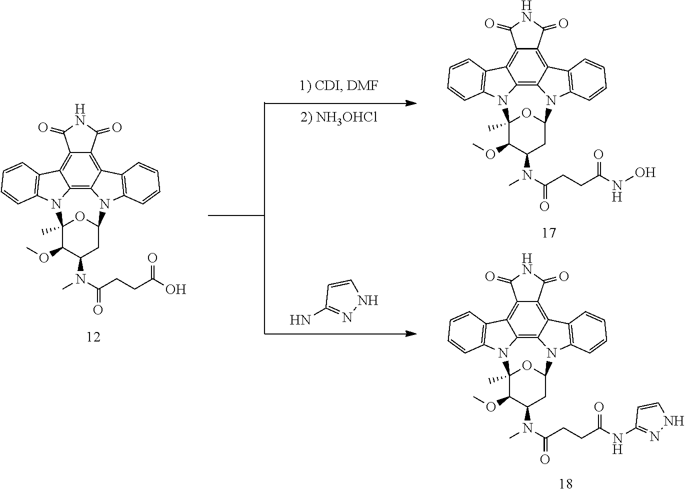 [SMILES: CO[C@@H]1[C@H](N(C)C(=O)CCC(=O)O)C[C@H]2O[C@]1(C)n1c3ccccc3c3c4c(c5c6ccccc6n2c5c31)C(=O)NC4=O ; Nc1cc[nH]n1 ; CO[C@@H]1[C@H](N(C)C(=O)CCC(=O)NO)C[C@H]2O[C@]1(C)n1c3ccccc3c3c4c(c5c6ccccc6n2c5c31)C(=O)NC4=O ; CO[C@@H]1[C@H](N(C)C(=O)CCC(=O)Nc2cc[nH]n2)C[C@H]2O[C@]1(C)n1c3ccccc3c3c4c(c5c6ccccc6n2c5c31)C(=O)NC4=O]

synthesis of compounds 1-11 using an acyl halide method: enabling the initial substrate to react with acyl chlorides with different groups respectively in a mixed environment of N,N-diisopropylethylamine (DIPEA) and chloroform, or N,N-diisopropylethylamine and anhydrous N,N-dimethylformamide (DMF) to obtain the compounds 1-11, respectively;
synthesis of compounds 12-15 using an acid anhydride method: enabling the initial substrate to react with different acid anhydrides respectively in a mixed environment of 4-dimethylaminopyridine (DMAP) and dimethyl sulfoxide (DMSO) to obtain the compounds 12-15, respectively;
synthesis of compound 16 using an onium salt-type condensing agent method: enabling the initial substrate to react with suberic acid in a mixed environment of N,N-diisopropylethylamine, N,N-dimethylformamide, and 2-(7-azabenzotriazolyl)-N,N,N′,N′-tetramethyluronium hexafluorophosphate (HATU) to obtain the compound 16;
synthesis of compound 17 using an N,N′-carbonyldiimidazole (CDI) condensing agent method: first enabling the compound 12 to react in a mixed environment of N,N′-carbonyldiimidazole (CDI) and N,N-dimethylformamide, and then adding hydroxylamine hydrochloride to continue the reaction to obtain the compound 17; and
enabling the compound 12 to react with 3-aminopyrazole in a mixed environment of N,N-diisopropylethylamine, N,N-dimethylformamide, and 2-(7-azabenzotriazolyl)-N,N,N′,N′-tetramethyluronium hexafluorophosphate (HATU) to obtain compound 18.

The above preparation method involves a total of 1-2 reaction steps and is easy to operate and implement.

Compared with the prior art, the present invention has the following beneficial effects.

The compounds disclosed in the present invention have significant inhibitory activity against a PIM-1 kinase and excellent kinase selectivity, and can be used for development of therapeutic drugs for related diseases or disorders caused by abnormal expression of the PIM-1 kinase.

The synthetic route of the compounds involved in the present invention is simple and easy to operate and implement, and has easily purchasable required reagents. The derivative compound 17 has a better inhibitory effect on the PIM-1 kinase than a positive drug TP-3654 at a clinical development stage, and has excellent kinase selectivity, thus having greater advantages in targeted and specific clinical therapy.

### DETAILED DESCRIPTION OF THE EMBODIMENTS

The present invention is further illustrated below in combination with specific embodiments.

It should be understood that these embodiments are merely used for illustrating the present invention and are not intended to limit the scope of the present invention. Operation methods without specific conditions in the following embodiments are generally carried out according to conventional conditions or according to conditions recommended by manufacturers.

### Example 1. Synthesis of Derivatives

Synthesis steps of compounds 1-8 are as follows.

10.0 mg (0.021 mmol) of 7-carbonylstaurosporine was weighed and added into a 25 mL reaction flask, 2 mL of chloroform was added for dissolution, and then 40 μL (0.230 mmol) of DIPEA and 40 μL (0.476 mmol) of propionyl chloride were added and stirred to carry out a reaction at 30° C. for 4 hours. A reaction progress was monitored by thin-layer chromatography.

After concentration under reduced pressure, preparation was performed by high performance liquid chromatography (HPLC) with 85% methanol-water-0.05% trifluoroacetic acid (TFA) as a mobile phase at a flow rate of 10 mL/min and a detection wavelength of 318 nm to obtain compound 1 (4.0 mg, yield: approximately 35.8%, t_{R}=18 min).

According to the above reaction, compound 2 (4.0 mg, yield: approximately 34.9%, t_{R}=17 min) was obtained by replacing the reactant with n-butyryl chloride and changing the mobile phase to 87% methanol-water-0.05% TFA at the flow rate of 10 mL/min and the detection wavelength of 318 nm; compound 3 (6.1 mg, yield: approximately 51.9%, t_{R}=20 min) was obtained by replacing the reactant with n-valeryl chloride and changing the mobile phase to 87% methanol-water-0.05% TFA at the flow rate of 10 mL/min and the detection wavelength of 318 nm; compound 4 (8.0 mg, yield: approximately 66.4%, t_{R}=18 min) was obtained by replacing the reactant with hexanoyl chloride and changing the mobile phase to 90% methanol-water-0.05% TFA at the flow rate of 10 mL/min and the detection wavelength of 318 nm; compound 5 (4.5 mg, yield: approximately 36.5%, t_{R}=18 min) was obtained by replacing the reactant with heptanoyl chloride and changing the mobile phase to 92% methanol-water-0.05% TFA at the flow rate of 10 mL/min and the detection wavelength of 318 nm; compound 6 (4.9 mg, yield: approximately 38.8%, t_{R}=19 min) was obtained by replacing the reactant with octanoyl chloride and changing the mobile phase to 93% methanol-water-0.05% TFA at the flow rate of 10 mL/min and the detection wavelength of 318 nm; compound 7 (4.7 mg, yield: approximately 41.2%, t_{R}=18 min) was obtained by replacing the reactant with cyclopropylformyl chloride and changing the mobile phase to 85-100% methanol-water-0.05% TFA for gradient elution at the flow rate of 10 mL/min and the detection wavelength of 318 nm; and compound 8 (5.6 mg, yield: approximately 45.6%, t_{R}=19 min) was obtained by replacing the reactant with 2-thiophenylformyl chloride and changing the mobile phase to 85-100% methanol-water-0.05% TFA for gradient elution at the flow rate of 10 mL/min and the detection wavelength of 318 nm.

Synthesis steps of compounds 9-11 are as follows.

10.0 mg (0.021 mmol) of 7-carbonylstaurosporine was weighed and added into a 25 mL reaction flask, 2 mL of anhydrous DMF was added for dissolution, and then 20 μL (0.115 mmol) of DIPEA and 7.8 mg (0.042 mmol) of piperonyloyl chloride were added and stirred to carry out a reaction at 30° C. for 4 hours. A reaction progress was monitored by thin-layer chromatography. After concentration under reduced pressure, preparation was performed by HPLC with 68% acetonitrile-water as a mobile phase at a flow rate of 10 mL/min and a detection wavelength of 318 nm to obtain compound 9 (6.1 mg, yield: approximately 46.6%, t_{R}=16 min).

According to the above reaction, compound 10 (6.1 mg, yield: approximately 52.1%, t_{R}=15 min) was obtained by replacing the reactant with 3-methylcrotonyl chloride and changing the mobile phase to 80% acetonitrile-water-0.05% TFA at the flow rate of 10 mL/min and the detection wavelength of 318 nm; and compound 11 (5.9 mg, yield: approximately 46.4%, t_{R}=19 min) was obtained by replacing the reactant with 3-phenylacryloyl chloride and changing the mobile phase to 80% acetonitrile-water-0.05% TFA at the flow rate of 10 mL/min and the detection wavelength of 318 nm.

Synthesis steps of compounds 12-15 are as follows.

10.0 mg (0.021 mmol) of 7-carbonylstaurosporine, 3.2 mg (0.032 mmol) of succinic anhydride, and 1.0 mg of DMAP were weighed and added into a 25 mL reaction flask, 3 mL of DMSO was added for dissolution, and then stirring was performed to carry out a reaction at 30° C. for 8 hours. A reaction progress was monitored by thin-layer chromatography. 50 mL of water was added, extraction was performed with an equal volume of ethyl acetate for 3 times to remove most of the DMSO, and an ethyl acetate phase was concentrated under reduced pressure. Then, preparation was performed by HPLC with 52% acetonitrile-water-0.05% TFA as a mobile phase at a flow rate of 10 mL/min and a detection wavelength of 318 nm to obtain compound 12 (5.5 mg, yield: approximately 45.5%, t_{R}=21 min).

According to the above reaction, compound 13 (3.0 mg, yield: approximately 24.2%, t_{R}=3⁴ min) was obtained by replacing the reactant with glutaric anhydride and changing the mobile phase to 50% acetonitrile-water-0.05% TFA at the flow rate of 10 mL/min and the detection wavelength of 318 nm; compound 14 (2.8 mg, yield: approximately 22.0%, t_{R}=21 min) was obtained by replacing the reactant with 3-thioglutaric anhydride and changing the mobile phase to 58% acetonitrile-water-0.05% TFA at the flow rate of 10 mL/min and the detection wavelength of 318 nm; and compound 15 (2.5 mg, yield: approximately 19.7%, t_{R}=19 min) was obtained by replacing the reactant with adipic anhydride and changing the mobile phase to 60% acetonitrile-water-0.05% TFA at the flow rate of 10 m/min and the detection wavelength of 318 nm.

Synthesis steps of compound 16 are as follows.

7.3 mg (0.042 mmol) of suberic acid and 11.8 mg (0.031 mmol) of HATU were weighed and added into a 25 mL reaction flask, 2 mL of DMF was added for dissolution, and then 20 μL (0.115 mmol) of DIPEA was added and stirred to carry out a reaction at 30° C. for 1 hour. Finally, 10.0 mg (0.021 mmol) of 7-carbonylstaurosporine was added and stirred to carry out a reaction at 65° C. for 8 hours. A reaction progress was monitored by thin-layer chromatography. After concentration under reduced pressure, preparation was performed by HPLC with 60% acetonitrile-water-0.05% TFA as a mobile phase at a flow rate of 10 mL/min and a detection wavelength of 318 nm to obtain compound 16 (3.2 mg, yield: approximately 24.2%, t_{R}=2⁷ min).

Synthesis steps of compound 17 are as follows.

15.0 mg (0.026 mmol) of the compound 12 and 8.4 mg (0.052 mmol) of CDI were weighed and added into a 25 mL reaction flask, 2 mL of DMF was added for dissolution, stirring was performed to carry out a reaction at 30° C. for 1 hour, and finally, 7.2 mg (0.104 mmol) of hydroxylamine hydrochloride was added and stirred to carry out a reaction at 30° C. for 8 hours. A reaction progress was monitored by thin-layer chromatography. After concentration under reduced pressure, preparation was performed by HPLC with 58% acetonitrile-water-0.05% TFA as a mobile phase at a flow rate of 10 mL/min and a detection wavelength of 318 nm to obtain compound 17 (2.6 mg, yield: approximately 14.0%, t_{R}=11 min).

Synthesis steps of compound 18 are as follows.

12.0 mg (0.021 mmol) of the compound 12 and 12.2 mg (0.032 mmol) of HATU were weighed and added into a 25 mL reaction flask, 2 mL of DMF was added for dissolution, then 20 L (0.115 mmol) of DIPEA was added and stirred to carry out a reaction at 30° C. for 1 hour, and finally, 2.0 mg (0.024 mmol) of 3-aminopyrazole was added and stirred to carry out a reaction at 65° C. for 8 hours. A reaction progress was monitored by thin-layer chromatography. After concentration under reduced pressure, preparation was performed by HPLC with 65% acetonitrile-water as a mobile phase at a flow rate of 10 mL/min and a detection wavelength of 318 nm to obtain compound 18 (2.0 mg, yield: approximately 12.4%, t_{R}=20 min).

### Example 2. Identification of Compounds

Compound 1: A yellow solid. HRESIMS found m/z 559.1957 [M+Na]⁺ (calcd for C₃₁H₂₈N₄O₅Na, 559.1957). ¹H NMR (600 MHz, DMSO-d₆): δ 11.14 (s, 1H), 9.26 (d, J=8.0 Hz, 1H), 9.09 (d, J=8.0 Hz, 1H), 8.04 (d, J=8.0 Hz, 1H), 7.73 (d, J=8.0 Hz, 1H), 7.62 (t, J=8.0 Hz, 1H), 7.56 (t, J=8.0 Hz, 1H), 7.43 (t, J=8.0 Hz, 1H), 7.40 (t, J=8.0 Hz, 1H), 7.06 (dd, J=8.8, 6.0 Hz, 1H), 5.07 (ddd, J=13.1, 4.6, 2.4 Hz, 1H), 4.26-4.23 (m, 1H), 2.80 (s, 3H), 2.75-2.69 (m, 1H), 2.66 (s, 3H), 2.39-2.35 (m, 5H), 2.28 (td, J=13.1, 6.0 Hz, 1H), 1.05 (t, J=7.3 Hz, 3H). ¹³C NMR (151 MHz, DMSO-d₆): δ 173.6, 171.2, 170.9, 139.8, 137.7, 130.6, 128.8, 127.2, 126.9, 125.0, 124.9, 122.8, 121.5, 120.9, 120.7, 120.7, 119.6, 116.1, 115.2, 113.6, 109.9, 95.0, 83.3, 82.5, 60.4, 47.8, 30.7, 29.2, 26.9, 26.4, 9.2.

Compound 2: A yellow solid. HRESIMS found m/z 551.2297 [M+H]⁺ (calcd for C₃₂H₃₁N₄O₅, 551.2294). ¹H NMR (600 MHz, DMSO-d₆): δ 11.13 (s, 1H), 9.26 (d, J=8.0 Hz, 1H), 9.09 (d, J=8.0 Hz, 1H), 8.04 (d, J=8.0 Hz, 1H), 7.73 (d, J=8.0 Hz, 1H), 7.61 (t, J=8.0 Hz, 1H), 7.56 (t, J=8.0 Hz, 1H), 7.45-7.38 (m, 2H), 7.05 (dd, J=8.8, 6.0 Hz, 1H), 5.06 (ddd, J=13.0, 4.6, 2.4 Hz, 1H), 4.26-4.23 (m, 1H), 2.80 (s, 3H), 2.75-2.69 (m, 1H), 2.67 (s, 3H), 2.37 (s, 3H), 2.33 (td, J=7.4, 4.2 Hz, 2H), 2.28 (td, J=13.0, 6.0 Hz, 1H), 1.58 (h, J=7.4 Hz, 2H), 0.93 (t, J=7.4 Hz, 3H). ¹³C NMR (151 MHz, DMSO-d₆): δ 172.8, 171.2, 170.9, 139.8, 137.7, 130.5, 128.7, 127.2, 126.9, 125.0, 124.9, 122.8, 121.4, 120.9, 120.7, 120.6, 119.6, 116.1, 115.2, 113.6, 109.9, 95.0, 83.3, 82.4, 60.4, 47.8, 35.0, 30.9, 29.2, 26.8, 18.0, 13.9.

Compound 3: A yellow solid. HRESIMS found m/z 565.2447 [M+H]⁺ (calcd for C₃₃H₃₃N₄O₅, 565.2451). ¹H NMR (600 MHz, DMSO-d₆): δ 11.13 (s, 1H), 9.26 (d, J=8.0 Hz, 1H), 9.08 (d, J=8.0 Hz, 1H), 8.03 (d, J=8.0 Hz, 1H), 7.72 (d, J=8.0 Hz, 1H), 7.61 (t, J=8.0 Hz, 1H), 7.55 (t, J=8.0 Hz, 1H), 7.45-7.37 (m, 2H), 7.05 (dd, J=8.8, 6.0 Hz, 1H), 5.05 (ddd, J=13.1, 4.6, 2.5 Hz, 1H), 4.25-4.22 (m, 1H), 2.80 (s, 3H), 2.74-2.68 (m, 1H), 2.66 (s, 3H), 2.37 (s, 3H), 2.34 (td, J=7.4, 2.1 Hz, 2H), 2.27 (td, J=13.1, 6.0 Hz, 1H), 1.53 (p, J=7.4 Hz, 2H), 1.37-1.29 (m, 2H), 0.89 (t, J=7.4 Hz, 3H). ¹³C NMR (151 MHz, DMSO-d₆): δ 173.0, 171.2, 170.9, 139.8, 137.6, 130.5, 128.7, 127.2, 126.9, 125.0, 124.9, 122.8, 121.5, 120.9, 120.7, 120.6, 119.6, 116.1, 115.2, 113.6, 109.8, 95.0, 83.3, 82.4, 60.4, 47.8, 32.8, 30.9, 29.2, 26.8, 26.7, 22.0, 14.0.

Compound 4: A yellow solid. HRESIMS found m/z 579.2612 [M+H]⁺ (calcd for C₃₄H₃₅N₄O₅, 579.2607). ¹H NMR (600 MHz, DMSO-d₆): δ 11.14 (s, 1H), 9.26 (d, J=8.0 Hz, 1H), 9.08 (d, J=8.0 Hz, 1H), 8.03 (d, J=8.0 Hz, 1H), 7.72 (d, J=8.0 Hz, 1H), 7.61 (t, J=8.0 Hz, 1H), 7.55 (t, J=8.0 Hz, 1H), 7.45-7.37 (m, 2H), 7.05 (dd, J=8.8, 6.0 Hz, 1H), 5.05 (ddd, J=13.1, 4.6, 2.4 Hz, 1H), 4.25-4.22 (m, 1H), 2.79 (s, 3H), 2.75-2.68 (m, 1H), 2.66 (s, 3H), 2.36 (s, 3H), 2.33 (td, J=7.2, 1.7 Hz, 2H), 2.27 (td, J=13.1, 6.0 Hz, 1H), 1.58-1.51 (m, 2H), 1.35-1.25 (m, 4H), 0.91-0.84 (m, 3H). ¹³C NMR (151 MHz, DMSO-d₆): δ 173.0, 171.2, 170.9, 139.8, 137.6, 130.5, 128.7, 127.2, 126.9, 125.0, 124.9, 122.8, 121.5, 120.9, 120.7, 120.6, 119.6, 116.1, 115.2, 113.6, 109.8, 95.0, 83.3, 82.4, 60.4, 47.8, 33.1, 31.1, 30.9, 29.2, 26.8, 24.3, 22.1, 14.0.

Compound 5: A yellow solid. HRESIMS found m/z 593.2764 [M+H]⁺ (calcd for C₃₅H₃₇N₄O₅, 593.2764). ¹H NMR (600 MHz, DMSO-d₆): δ 11.14 (s, 1H), 9.26 (d, J=8.0 Hz, 1H), 9.09 (d, J=8.0 Hz, 1H), 8.04 (d, J=8.0 Hz, 1H), 7.73 (d, J=8.0 Hz, 1H), 7.62 (t, J=8.0 Hz, 1H), 7.56 (t, J=8.0 Hz, 1H), 7.46-7.37 (m, 2H), 7.06 (dd, J=8.8, 6.1 Hz, 1H), 5.05 (ddd, J=13.1, 4.6, 2.4 Hz, 1H), 4.26-4.22 (m, 1H), 2.80 (s, 3H), 2.75-2.69 (m, 1H), 2.68 (s, 3H), 2.37 (s, 3H), 2.34 (td, J=7.2, 2.0 Hz, 2H), 2.27 (td, J=13.1, 6.1 Hz, 1H), 1.54 (p, J=7.2 Hz, 2H), 1.34-1.20 (m, 6H), 0.90-0.83 (m, 3H). ¹³C NMR (151 MHz, DMSO-d₆): δ 173.0, 171.2, 170.9, 139.8, 137.7, 130.5, 128.7, 127.2, 126.9, 125.0, 124.9, 122.8, 121.4, 120.9, 120.7, 120.6, 119.6, 116.1, 115.2, 113.6, 109.9, 95.0, 83.3, 82.4, 60.4, 47.8, 33.1, 31.3, 30.9, 29.2, 28.6, 26.8, 24.5, 22.2, 14.1.

Compound 6: A yellow solid. HRESIMS found m/z 607.2926 [M+H]⁺ (calcd for C₃₆H₃₉N₄O₅ 607.2920). ¹H NMR (600 MHz, DMSO-d₆): δ 11.14 (s, 1H), 9.26 (d, J=8.0 Hz, 1H), 9.09 (d, J=8.0 Hz, 1H), 8.03 (d, J=8.0 Hz, 1H), 7.73 (d, J=8.0 Hz, 1H), 7.61 (t, J=8.0 Hz, 1H), 7.56 (t, J=8.0 Hz, 1H), 7.45-7.37 (m, 2H), 7.05 (dd, J=8.8, 6.1 Hz, 1H), 5.05 (ddd, J=13.1, 4.6, 2.5 Hz, 1H), 4.26-4.21 (m, 1H), 2.80 (s, 3H), 2.74-2.68 (m, 1H), 2.67 (s, 3H), 2.37 (s, 3H), 2.35-2.31 (m, 2H), 2.27 (td, J=13.1, 6.1 Hz, 1H), 1.54 (p, J=7.2 Hz, 2H), 1.31-1.22 (m, 8H), 0.88-0.82 (m, 3H). ¹³C NMR (151 MHz, DMSO-d₆): δ 173.0, 171.2, 170.9, 139.8, 137.7, 130.5, 128.7, 127.2, 126.9, 125.0, 124.9, 122.8, 121.4, 120.9, 120.7, 120.6, 119.6, 116.1, 115.2, 113.6, 109.9, 95.0, 83.3, 82.4, 60.4, 47.8, 33.1, 31.3, 30.9, 29.2, 28.9, 28.7, 26.8, 24.6, 22.2, 14.1.

Compound 7: A yellow solid. HRESIMS found m/z 549.2138 [M+H]⁺ (calcd for C₃₂H₂₉N₄O₅ 549.2138). ¹H NMR (600 MHz, DMSO-d₆): δ 11.13 (s, 1H), 9.26 (d, J=8.0 Hz, 1H), 9.09 (d, J=8.0 Hz, 1H), 8.02 (d, J=8.0 Hz, 1H), 7.73 (d, J=8.0 Hz, 1H), 7.62 (t, J=8.0 Hz, 1H), 7.55 (t, J=8.0 Hz, 1H), 7.46-7.37 (m, 2H), 7.05 (dd, J=8.8, 6.1 Hz, 1H), 5.02 (ddd, J=13.0, 4.5, 2.5 Hz, 1H), 4.28-4.23 (m, 1H), 2.98 (s, 3H), 2.79-2.71 (m, 1H), 2.70 (s, 3H), 2.35 (s, 3H), 2.30 (td, J=13.0, 6.1 Hz, 1H), 1.99-1.91 (m, 1H), 0.93-0.73 (m, 4H). ¹³C NMR (151 MHz, DMSO-d₆): δ 173.3, 171.1, 170.9, 139.9, 137.7, 130.5, 128.7, 127.2, 126.8, 124.9, 124.9, 122.8, 121.4, 120.9, 120.7, 120.6, 119.6, 116.1, 115.2, 113.6, 109.8, 95.0, 83.2, 82.4, 60.4, 48.3, 31.0, 29.2, 26.8, 11.3, 7.5, 7.1.

Compound 8: A yellow solid. HRESIMS found m/z 591.1694 [M+H]⁺ (calcd for C₃₃H₂₇N₄O₅S 591.1702). ¹H NMR (600 MHz, DMSO-d₆): δ 11.14 (s, 1H), 9.27 (d, J=8.0 Hz, 1H), 9.09 (d, J=8.0 Hz, 1H), 8.06 (d, J=8.0 Hz, 1H), 7.83 (d, J=5.0 Hz, 1H), 7.73 (d, J=8.0 Hz, 1H), 7.65-7.54 (m, 3H), 7.46-7.38 (m, 2H), 7.18 (s, 1H), 7.13-7.03 (m, 1H), 5.01 (s, 1H), 4.48 (s, 1H), 3.06 (s, 3H), 2.96-2.87 (m, 1H), 2.68 (s, 3H), 2.46-2.27 (m, 4H). ¹³C NMR (151 MHz, DMSO-d₆): δ 171.1, 170.9, 164.1, 139.8, 137.9, 137.7, 130.6, 130.4, 129.7, 128.8, 127.5, 127.2, 126.9, 125.0, 125.0, 122.8, 121.5, 121.0, 120.7, 120.7, 119.6, 116.1, 115.2, 113.5, 109.8, 95.0, 83.4, 82.4, 60.3, 48.7, 33.6, 29.1, 27.0.

Compound 9: An orange solid. HRESIMS found m/z 629.2032 [M+H]⁺ (calcd for C₃₆H₂₉N₄O₇ 629.2036). ¹H NMR (600 MHz, Chloroform-d): δ 9.39 (d, J=8.0 Hz, 1H), 9.25 (d, J=8.0 Hz, 1H), 7.77 (d, J=8.0 Hz, 1H), 7.61 (t, J=8.0 Hz, 1H), 7.57-7.52 (m, 2H), 7.47 (t, J=8.0 Hz, 1H), 7.44 (t, J=8.0 Hz, 1H), 7.32 (d, J=8.0 Hz, 1H), 6.96 (s, 1H), 6.93 (s, 1H), 6.86 (s, 1H), 6.78 (s, 1H), 6.03 (s, 2H), 5.21 (s, 1H), 4.22 (s, 1H), 2.92 (s, 3H), 2.82 (s, 1H), 2.78-2.69 (m, 1H), 2.56 (s, 3H), 2.44 (s, 3H). ¹³C NMR (151 MHz, Chloroform-d): δ 171.9, 169.9, 169.7, 149.2, 147.8, 139.5, 137.9, 131.6, 130.2, 129.4, 127.2, 127.1, 126.5, 126.3, 123.8, 122.6, 121.9, 121.4, 121.3, 121.0, 119.6, 117.4, 116.7, 116.5, 113.4, 111.7, 108.4, 101.7, 94.9, 84.9, 82.6, 60.5, 49.8, 34.5, 29.0, 28.1.

Compound 10: A yellow solid. HRESIMS found m/z 563.2291 [M+H]⁺ (calcd for C₃₃H₃₁N₄O₅ 563.2294). ¹H NMR (600 MHz, DMSO-d₆): δ 11.14 (s, 1H), 9.26 (d, J=8.0 Hz, 1H), 9.09 (d, J=8.0 Hz, 1H), 8.05 (d, J=8.0 Hz, 1H), 7.74 (d, J=8.0 Hz, 1H), 7.62 (t, J=8.0 Hz, 1H), 7.56 (t, J=8.0 Hz, 1H), 7.46-7.38 (m, 2H), 7.07 (dd, J=8.7, 6.2 Hz, 1H), 5.97 (s, 1H), 5.09-4.98 (m, 1H), 4.31 (s, 1H), 2.82 (s, 3H), 2.79-2.72 (m, 1H), 2.70 (s, 3H), 2.39 (s, 3H), 2.29 (td, J=13.0, 6.2 Hz, 1H), 1.94 (s, 3H), 1.83 (s, 3H). ¹³C NMR (151 MHz, DMSO-d₆): δ 171.1, 170.9, 167.8, 146.9, 139.9, 137.7, 130.5, 128.7, 127.2, 126.8, 124.9, 124.9, 122.8, 121.4, 120.9, 120.7, 120.7, 119.6, 118.2, 116.1, 115.2, 113.7, 109.9, 95.0, 83.3, 82.4, 60.4, 47.7, 31.6, 29.3, 26.8, 26.1, 20.0.

Compound 11: A yellow solid. HRESIMS found m/z 611.2292 [M+H]⁺ (calcd for C₃₇H₃₁N₄O₅ 611.2294). ¹H NMR (600 MHz, DMSO-d₆): δ 11.15 (s, 1H), 9.26 (d, J=8.0 Hz, 1H), 9.10 (d, J=8.0 Hz, 1H), 8.05 (d, J=8.0 Hz, 1H), 7.78-7.71 (m, 3H), 7.65-7.60 (m, 2H), 7.56 (t, J=8.0 Hz, 1H), 7.47-7.37 (m, 5H), 7.25 (d, J=15.4 Hz, 1H), 7.10 (dd, J=8.8, 6.1 Hz, 1H), 5.18 (ddd, J=13.0, 4.7, 2.5 Hz, 1H), 4.36 (s, 1H), 3.03 (s, 3H), 2.83-2.78 (m, 1H), 2.70 (s, 3H), 2.41 (s, 3H), 2.37 (td, J=13.0, 6.1 Hz, 1H). ¹³C NMR (151 MHz, DMSO-d₆): δ 171.2, 170.9, 166.4, 142.3, 139.8, 137.7, 135.1, 130.6, 129.8, 129.0, 128.9, 128.8, 128.2, 128.2, 127.2, 126.9, 125.0, 124.9, 122.8, 121.5, 120.9, 120.7, 120.7, 119.6, 118.7, 116.1, 115.2, 113.6, 109.9, 95.0, 83.3, 82.4, 60.4, 48.4, 31.2, 29.2, 26.8.

Compound 12: A yellow solid. HRESIMS found m/z 581.2037 [M+H]⁺ (calcd for C₃₂H₂₉N₄O₇ 581.2036). ¹H NMR (600 MHz, DMSO-d₆): δ 12.09 (s, 1H), 11.14 (s, 1H), 9.26 (d, J=8.0 Hz, 1H), 9.09 (d, J=8.0 Hz, 1H), 8.03 (d, J=8.0 Hz, 1H), 7.75 (d, J=8.0 Hz, 1H), 7.62 (t, J=8.0 Hz, 1H), 7.56 (t, J=8.0 Hz, 1H), 7.45-7.38 (m, 2H), 7.09-7.04 (m, 1H), 5.01 (ddd, J=13.1, 4.5, 2.5 Hz, 1H), 4.23 (s, 1H), 2.83 (s, 3H), 2.75-2.67 (m, 4H), 2.63-2.55 (m, 3H), 2.46-2.43 (m, 1H), 2.36 (s, 3H), 2.29 (td, J=13.1, 6.2 Hz, 1H). ¹³C NMR (151 MHz, DMSO-d₆): δ 174.1, 172.0, 171.1, 170.9, 139.8, 137.7, 130.5, 128.7, 127.2, 126.9, 125.0, 124.9, 122.8, 121.4, 120.9, 120.7, 120.6, 119.6, 116.1, 115.2, 113.6, 109.8, 95.0, 83.2, 82.4, 60.4, 48.1, 30.8, 29.3, 29.0, 28.4, 26.8.

Compound 13: A yellow solid. HRESIMS found m/z 595.2189 [M+H]⁺ (calcd for C₃₃H₃₁N₄O₇ 595.2193). ¹H NMR (600 MHz, DMSO-d₆): δ 11.13 (s, 1H), 9.26 (d, J=8.0 Hz, 1H), 9.09 (d, J=8.0 Hz, 1H), 8.04 (d, J=8.0 Hz, 1H), 7.73 (d, J=8.0 Hz, 1H), 7.62 (t, J=8.0 Hz, 1H), 7.56 (t, J=8.0 Hz, 1H), 7.43 (t, J=8.0 Hz, 1H), 7.40 (t, J=8.0 Hz, 1H), 7.06 (dd, J=8.8, 6.1 Hz, 1H), 5.06 (ddd, J=13.1, 4.5, 2.4 Hz, 1H), 4.26 (s, 1H), 2.80 (s, 3H), 2.76-2.70 (m, 1H), 2.68 (s, 3H), 2.42-2.38 (m, 2H), 2.38 (s, 3H), 2.31 (t, J=7.4 Hz, 2H), 2.27 (td, J=13.1, 6.1 Hz, 1H), 1.78 (p, J=7.4 Hz, 2H). ¹³C NMR (151 MHz, DMSO-d₆): δ 174.4, 172.5, 171.2, 170.9, 139.8, 137.6, 130.6, 128.7, 127.2, 126.9, 125.0, 124.9, 122.8, 121.4, 120.9, 120.7, 120.6, 119.6, 116.1, 115.2, 113.6, 109.8, 95.0, 83.3, 82.4, 60.4, 47.8, 33.0, 32.3, 30.8, 29.2, 26.8, 20.1.

Compound 14: A yellow solid. HRESIMS found m/z 613.1761 [M+H]⁺ (calcd for C₃₂H₂₉N₄O₇S 613.1757). ¹H NMR (600 MHz, DMSO-d₆): δ 11.13 (s, 1H), 9.26 (d, J=8.0 Hz, 1H), 9.09 (d, J=8.0 Hz, 1H), 8.05 (d, J=8.0 Hz, 1H), 7.73 (d, J=8.0 Hz, 1H), 7.62 (t, J=8.0 Hz, 1H), 7.57 (t, J=8.0 Hz, 1H), 7.46-7.38 (m, 2H), 7.07 (dd, J=8.8, 6.0 Hz, 1H), 5.00 (ddd, J=13.0, 4.5, 2.4 Hz, 1H), 4.26 (s, 1H), 3.66-3.56 (m, 2H), 3.45-3.37 (m, 2H), 2.86 (s, 3H), 2.80-2.72 (m, 1H), 2.70 (s, 3H), 2.38 (s, 3H), 2.29 (td, J=13.0, 6.0 Hz, 1H). ¹³C NMR (151 MHz, DMSO-d₆): δ 171.2, 171.1, 170.9, 169.1, 139.8, 137.7, 130.5, 128.7, 127.2, 126.9, 125.0, 125.0, 122.7, 121.4, 120.9, 120.7, 120.6, 119.6, 116.1, 115.2, 113.5, 109.9, 95.0, 83.1, 82.4, 60.4, 48.4, 34.2, 33.3, 31.4, 29.2, 26.5.

Compound 15: A yellow solid. HRESIMS found m/z 609.2347 [M+H]⁺ (calcd for C₃₄H₃₃N₄O₇ 609.2349). ¹H NMR (600 MHz, Methanol-d₄): δ 9.25 (d, J=8.0 Hz, 1H), 9.04 (d, J=8.0 Hz, 1H), 7.83 (d, J=8.0 Hz, 1H), 7.53-7.48 (m, 2H), 7.39 (d, J=8.0 Hz, 1H), 7.34-7.29 (m, 2H), 6.76 (dd, J=9.4, 4.4 Hz, 1H), 5.15 (ddd, J=13.2, 5.6, 1.8 Hz, 1H), 4.01 (s, 1H), 2.86 (s, 3H), 2.74-2.67 (m, 1H), 2.46-2.41 (m, 3H), 2.40 (s, 3H), 2.37 (s, 3H), 2.34 (t, J=6.7 Hz, 2H), 1.71-1.63 (m, 4H). ¹³C NMR (151 MHz, Methanol-d₄): δ 177.2, 175.8, 172.4, 172.2, 140.7, 139.0, 132.4, 130.7, 127.8, 127.7, 126.7, 126.5, 124.6, 123.2, 121.8, 121.6, 121.5, 120.4, 117.9, 116.8, 113.2, 109.7, 96.0, 85.5, 83.8, 60.5, 49.8, 34.5, 34.3, 31.6, 29.1, 28.5, 25.5, 25.4.

Compound 16: A yellow solid. HRESIMS found m/z 637.2671 [M+H]⁺ (calcd for C₃₆H₃₇N₄O₇ 637.2662). ¹H NMR (600 MHz, DMSO-d₆): δ 11.96 (s, 1H), 11.13 (s, 1H), 9.26 (d, J=8.0 Hz, 1H), 9.09 (d, J=8.0 Hz, 1H), 8.04 (d, J=8.0 Hz, 1H), 7.73 (d, J=8.0 Hz, 1H), 7.62 (t, J=8.0 Hz, 1H), 7.56 (t, J=8.0 Hz, 1H), 7.43 (t, J=8.0 Hz, 1H), 7.40 (t, J=8.0 Hz, 1H), 7.06 (dd, J=8.6, 6.3 Hz, 1H), 5.06 (ddd, J=13.5, 4.4, 2.4 Hz, 1H), 4.25 (s, 1H), 2.81 (s, 3H), 2.75-2.69 (m, 1H), 2.68 (s, 3H), 2.37 (s, 3H), 2.36-2.32 (m, 2H), 2.29-2.24 (m, 1H), 2.21 (t, J=7.3 Hz, 2H), 1.59-1.53 (m, 2H), 1.53-1.47 (m, 2H), 1.35-1.25 (m, 4H). ¹³C NMR (151 MHz, DMSO-d₆): δ 174.6, 172.9, 171.2, 170.9, 139.9, 137.7, 130.5, 128.7, 127.2, 126.9, 125.0, 124.9, 122.8, 121.4, 120.9, 120.7, 120.6, 119.6, 116.1, 115.2, 113.5, 109.8, 95.0, 83.3, 82.4, 60.4, 47.8, 33.7, 33.0, 30.9, 29.2, 28.5, 28.5, 26.8, 24.5, 24.4.

Compound 17: A yellow solid. HRESIMS found m/z 596.2145 [M+H]⁺ (calcd for C₃₂H₃₀N₅O₇ 596.2145). ¹H NMR (600 MHz, DMSO-d₆): δ 11.14 (s, 1H), 10.43 (d, J=1.4 Hz, 1H), 9.26 (d, J=8.0 Hz, 1H), 9.09 (d, J=8.0 Hz, 1H), 8.70 (d, J=1.4 Hz, 1H), 8.03 (d, J=8.0 Hz, 1H), 7.74 (d, J=8.0 Hz, 1H), 7.62 (t, J=8.0 Hz, 1H), 7.56 (t, J=8.0 Hz, 1H), 7.46-7.37 (m, 2H), 7.06 (dd, J=8.8, 6.0 Hz, 1H), 5.03 (ddd, J=13.5, 4.6, 2.4 Hz, 1H), 4.24-4.18 (m, 1H), 2.82 (s, 3H), 2.76-2.66 (m, 4H), 2.64-2.55 (m, 3H), 2.36 (s, 3H), 2.32-2.25 (m, 2H). ¹³C NMR (151 MHz, DMSO-d₆): δ 172.0, 171.2, 170.9, 168.7, 139.8, 137.7, 130.6, 128.7, 127.2, 126.9, 125.0, 124.9, 122.8, 121.5, 121.0, 120.7, 120.7, 119.6, 116.1, 115.2, 113.5, 109.9, 95.0, 83.3, 82.4, 60.4, 48.0, 30.8, 29.2, 28.5, 27.4, 26.8.

Compound 18: A yellow solid. HRESIMS found m/z 646.2413 [M+H]⁺ (calcd for C₃₅H₃₂N₇O₆ 646.2414). ¹H NMR (600 MHz, Methanol-d₄): δ 9.28 (d, J=8.0 Hz, 1H), 9.08 (d, J=8.0 Hz, 1H), 7.83 (d, J=8.0 Hz, 1H), 7.58-7.49 (m, 3H), 7.43 (d, J=8.0 Hz, 1H), 7.34 (t, J=8.0 Hz, 2H), 6.80 (dd, J=9.1, 4.3 Hz, 1H), 6.51 (s, 1H), 5.15 (dd, J=13.4, 5.5 Hz, 1H), 4.07 (s, 1H), 2.93 (s, 3H), 2.81-2.70 (m, 5H), 2.50-2.45 (m, 1H), 2.45-2.43 (m, 3H), 2.40 (s, 3H). ¹³C NMR (151 MHz, Methanol-d₄): δ 174.8, 172.6, 172.4, 172.3, 154.7, 140.8, 139.1, 132.4, 130.8, 127.9, 127.7, 126.7, 126.5, 124.7, 123.3, 121.9, 121.7, 121.5, 121.1, 120.5, 117.9, 116.9, 113.3, 109.8, 96.1, 92.7, 85.4, 83.9, 60.5, 49.9, 31.7, 31.5, 29.6, 29.2, 28.4.

### Example 3. PIM-1 Kinase Inhibitory Activity and Kinase Selectivity Assay of Compounds

Inhibitory activities against PIM-1, TRKA, TRKB, FLT3, PKCδ, PKCε, PKCη, PAK4, PLK1, ROCK2, and IKKβ kinases were all determined using an HTRF®KinEASE™ kinase assay kit based on a time-resolved fluorescence technology. 7-carbonylstaurosporine (7OSTA) and TP-3654 were used as positive controls, and 3 duplicate wells were set in parallel. Specific operation steps are as follows.
\textcircled{1} A kinase buffer was formulated and then used to formulate compounds to be tested, a positive drug, a substrate, kinases, and adenosine triphosphate (ATP).
   \textcircled{2} 2 μL of the substrate, 2 μL of the kinases (the kinases were not added in a negative control group), 4 μL of the compounds to be tested or the positive drug (4 μL of a buffer was added in a positive control group, and 6 μL of a buffer was added in the negative control group), and 2 μL of the ATP were sequentially added into each well of a 384-well microplate, and the microplate was sealed with a membrane and then centrifuged for uniform mixing.
\textcircled{3} According to different types of the kinases, incubation was performed in an incubator at 37° C. for a specific time.
\textcircled{4} A detection solution was formulated using XL-665, an antibody, and a detection buffer, 10 L of the detection solution was added into each well, the microplate was sealed with a membrane and then centrifuged for uniform mixing, and standing was performed at room temperature for 1 hour.
\textcircled{5} Fluorescence value intensities F1 and F2 at wavelengths of 620 nm and 665 nm were measured using a multifunctional microplate tester, respectively. A signal ratio was calculated as F2/F1×10000, and then an IC₅₀ value was calculated using Graphpad Prism 8 software.

TABLE 1
PIM-1 kinase inhibitory activity of compounds
1-18 and positive controls (IC₅₀/nM)

| Compound | PIM-1 |
|---|---|
| 1 | 41.0 |
| 2 | 83.0 |
| 3 | >1000 |
| 4 | >1000 |
| 5 | >1000 |
| 6 | >1000 |
| 7 | 444.7 |
| 8 | 52.4 |
| 9 | >1000 |
| 10 | 81.4 |
| 11 | >1000 |
| 12 | 42.9 |
| 13 | 39.1 |
| 14 | 53.8 |
| 15 | 61.1 |
| 16 | 65.9 |
| 17 | 5.9 |
| 18 | 32.4 |
| 7OSTA (control) | 5.4 |
| TP-3654 (control) | 31.1 |

TABLE 2
Kinase selectivity of compound 17 and 7OSTA (IC₅₀/nM)

| Kinase | Compound 17 | 7OSTA |
|---|---|---|
| PIM-1 | 5.9 | 5.4 |
| TRKA | 4.0 | 0.4 |
| FLT3 | 5.3 | 15.3 |
| PKCδ | 10.4 | 1.4 |
| TRKB | 13.6 | 2.4 |
| PKCε | 69.0 | 2.3 |
| PKCη | 73.2 | 2.1 |
| PAK4 | 240.2 | 37.0 |
| PLK1 | 420.9 | 308.1 |
| ROCK2 | 561.7 | 35.9 |
| IKKβ | >1000 | >1000 |

Results show that most of the 7-carbonylstaurosporine derivatives synthesized in the present invention have good inhibitory effects on the PIM-1 kinase, and particularly, the compound 17 has a significant inhibitory effect on the PIM-1 kinase and has an IC₅₀ of 5.9 nM, thus having a superior effect than the positive control drug TP-3654 (with an IC₅₀ of 31.1 nM). In addition, by testing the inhibitory activities of the compound 17 and the 7OSTA against the TRKA, TRKB, FLT3, PKCδ, PKCε, PKCη, PAK4, PLK1, ROCK2, and IKKβ kinases, it is found that the compound 17 has an excellent selective inhibition ability against the PIM-1 kinase, indicating that the compound can be used for further developing a PIM-1 selective inhibitor.

Finally, it should also be noted that the terms “include”, “comprise”, or any other variants thereof are intended to cover non-exclusive inclusions, such that a process, a method, an article, or an apparatus that includes a list of elements not only includes those elements, but also includes other elements not expressly listed, or further includes elements inherent to such process, method, article, or apparatus.

Although preferred embodiments of the present invention have been described, those skilled in the art, once understanding the basic creative concept, may make further alterations and modifications to these embodiments. Therefore, the appended claims are intended to be construed to include the preferred embodiments as well as all alterations and modifications falling within the scope of the present invention.

It is apparent that those skilled in the art can make various modifications and variations to the present invention without departing from the spirit and scope of the present invention. Thus, if such modifications and variations of the present invention fall within the scope of the claims and their equivalent technologies of the present invention, the present invention is also intended to encompass these modifications and variations.

1. A 7-carbonylstaurosporine derivative or a pharmaceutically acceptable salt thereof, wherein the 7-carbonylstaurosporine derivative is selected from any one of the following compounds 4-18:

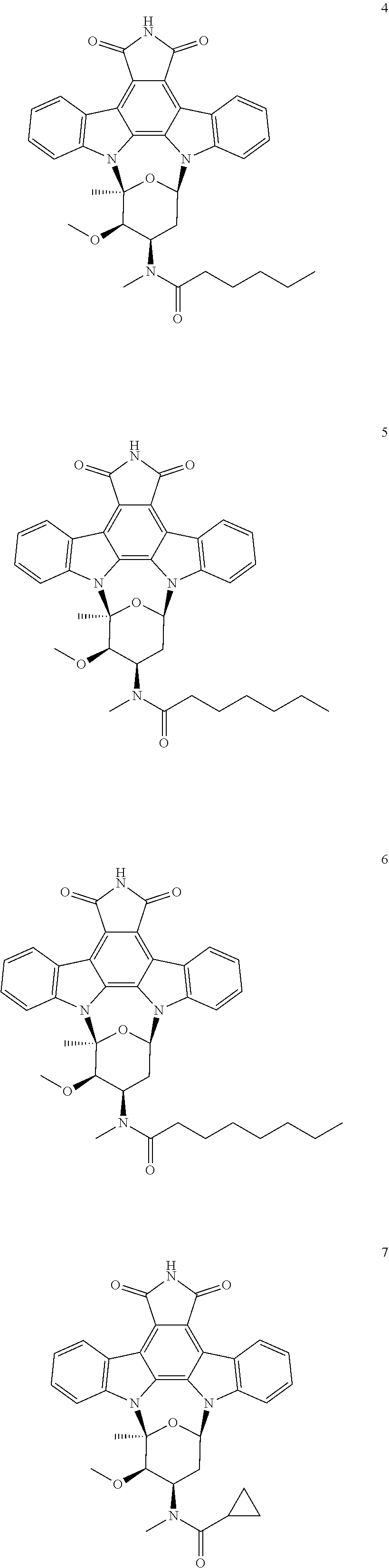 [SMILES: CCCCCC(=O)N(C)[C@@H]1C[C@H]2O[C@@](C)([C@@H]1OC)n1c3ccccc3c3c4c(c5c6ccccc6n2c5c31)C(=O)NC4=O ; CCCCCCC(=O)N(C)[C@@H]1C[C@H]2O[C@@](C)([C@@H]1OC)n1c3ccccc3c3c4c(c5c6ccccc6n2c5c31)C(=O)NC4=O ; CCCCCCCC(=O)N(C)[C@@H]1C[C@H]2O[C@@](C)([C@@H]1OC)n1c3ccccc3c3c4c(c5c6ccccc6n2c5c31)C(=O)NC4=O ; CO[C@@H]1[C@H](N(C)C(=O)C2CC2)C[C@H]2O[C@]1(C)n1c3ccccc3c3c4c(c5c6ccccc6n2c5c31)C(=O)NC4=O]

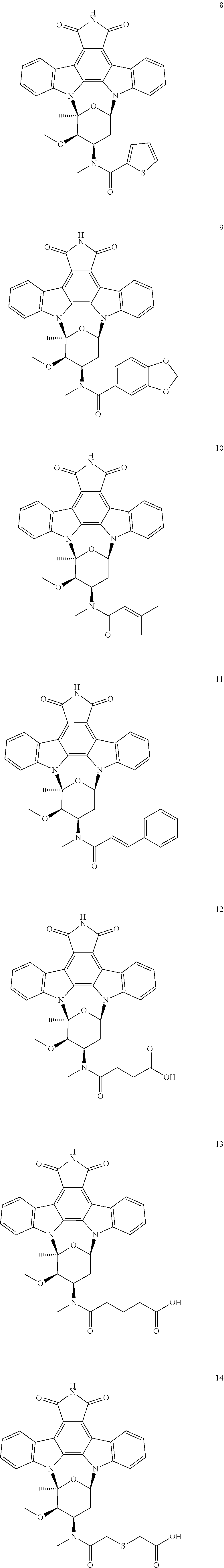

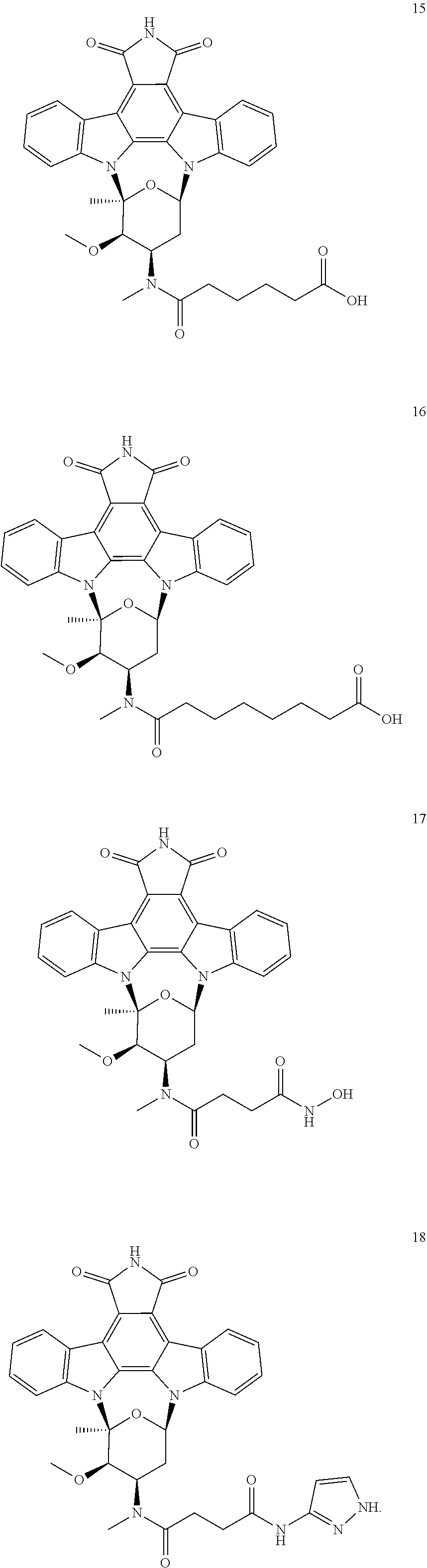 [SMILES: CO[C@@H]1[C@H](N(C)C(=O)CCCCC(=O)O)C[C@H]2O[C@]1(C)n1c3ccccc3c3c4c(c5c6ccccc6n2c5c31)C(=O)NC4=O ; CO[C@@H]1[C@H](N(C)C(=O)CCCCCCC(=O)O)C[C@H]2O[C@]1(C)n1c3ccccc3c3c4c(c5c6ccccc6n2c5c31)C(=O)NC4=O ; CO[C@@H]1[C@H](N(C)C(=O)CCC(=O)NO)C[C@H]2O[C@]1(C)n1c3ccccc3c3c4c(c5c6ccccc6n2c5c31)C(=O)NC4=O ; CO[C@@H]1[C@H](N(C)C(=O)CCC(=O)Nc2cc[nH]n2)C[C@H]2O[C@]1(C)n1c3ccccc3c3c4c(c5c6ccccc6n2c5c31)C(=O)NC4=O]

2. A pharmaceutical composition, comprising the 7-carbonylstaurosporine derivative or a pharmaceutically acceptable salt thereof according to claim 1, and one or more pharmaceutically acceptable carriers, diluents, or excipients.

3. A method of inhibiting a PIM-1 kinase in a subject comprising the step of administering to the subject the pharmaceutical composition according to claim 2.

4. A method of treating and/or preventing a disease associated with abnormal activity of a PIM-1 kinase comprising the step of administering to a subject a drug comprising the 7-carbonylstaurosporine derivative or a pharmaceutically acceptable salt thereof according to claim 1.

5. The method according to claim 4, wherein the disease is cancer.

6. The method according to claim 5, wherein the cancer is leukemia, lymphoma, multiple myeloma, breast cancer, endometrial cancer, ovarian cancer, vaginal cancer, carcinoma of fallopian tube, cervical cancer, renal cancer, bladder cancer, urothelial carcinoma, urethral carcinoma, prostate cancer, testicular cancer, colorectal cancer, sarcoma, bone cancer, myxoma, rhabdomyoma, leiomyoma, fibroma, lipoma, teratoma, pharyngolaryngeal cancer, nasopharyngeal carcinoma, oral cancer, lung cancer, alveolar carcinoma, mesothelioma, carcinoma of small intestine, gastric cancer, esophageal cancer, pancreatic cancer, liver cancer, cholangiocarcinoma, neurofibroma, neuroglioma, neuroblastoma, neuroblastoma, melanoma, skin cancer, basal cell carcinoma, squamous cell carcinoma, thyroid cancer, head and neck cancer, salivary gland cancer, or gastrointestinal stromal tumor.

7. The method according to claim 6, wherein the colorectal cancer is colon cancer or rectal cancer.

8. The method according to claim 6, wherein the sarcoma is chondrosarcoma.

9. A method for preparing the 7-carbonylstaurosporine derivative according to claim 1, comprising the following synthetic route with 7-carbonylstaurosporine as an initial substrate: 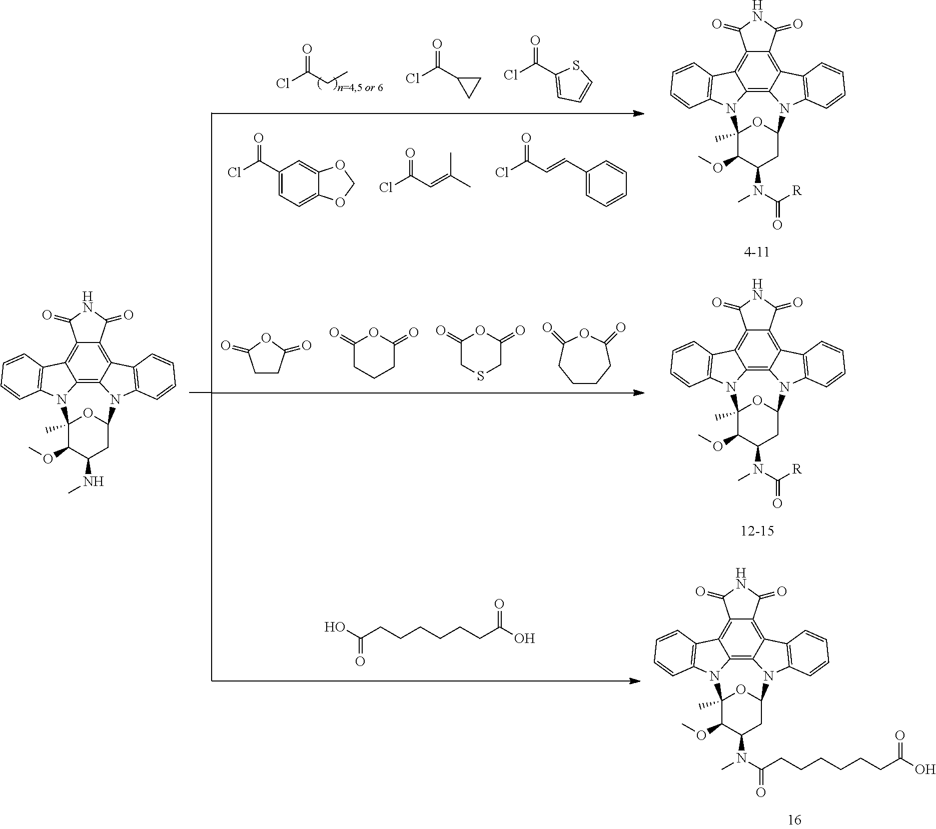 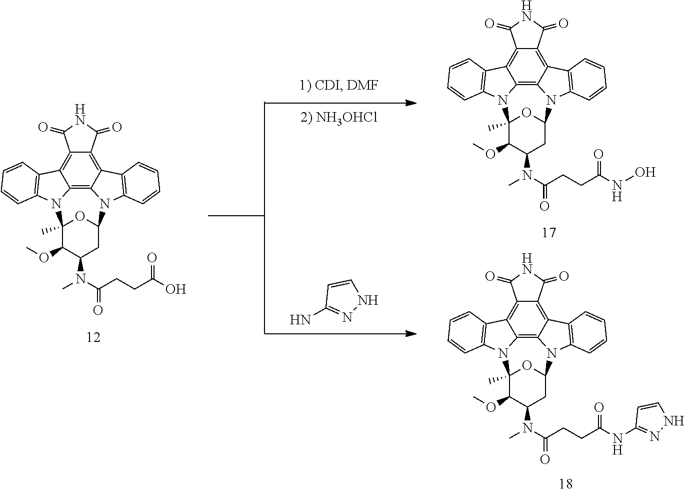 [SMILES: CO[C@@H]1[C@H](N(C)C(=O)CCC(=O)O)C[C@H]2O[C@]1(C)n1c3ccccc3c3c4c(c5c6ccccc6n2c5c31)C(=O)NC4=O ; Nc1cc[nH]n1 ; CO[C@@H]1[C@H](N(C)C(=O)CCC(=O)NO)C[C@H]2O[C@]1(C)n1c3ccccc3c3c4c(c5c6ccccc6n2c5c31)C(=O)NC4=O ; CO[C@@H]1[C@H](N(C)C(=O)CCC(=O)Nc2cc[nH]n2)C[C@H]2O[C@]1(C)n1c3ccccc3c3c4c(c5c6ccccc6n2c5c31)C(=O)NC4=O]
synthesis of compounds 4-11 using an acyl halide method: enabling the initial substrate to react with acyl chlorides with different groups respectively in a mixed environment of N,N-diisopropylethylamine and chloroform, or N,N-diisopropylethylamine and anhydrous N,N-dimethylformamide to obtain the compounds 4-11, respectively;
synthesis of compounds 12-15 using an acid anhydride method: enabling the initial substrate to react with different acid anhydrides respectively in a mixed environment of 4-dimethylaminopyridine and dimethyl sulfoxide to obtain the compounds 12-15, respectively;
synthesis of compound 16 using an onium salt-type condensing agent method: enabling the initial substrate to react with suberic acid in a mixed environment of N,N-diisopropylethylamine, N,N-dimethylformamide, and 2-(7-azabenzotriazolyl)-N,N,N′,N′-tetramethyluronium hexafluorophosphate to obtain the compound 16;
synthesis of compound 17 using an N,N′-carbonyldiimidazole (CDI) condensing agent method: first enabling the compound 12 to react in a mixed environment of N,N′-carbonyldiimidazole and N,N-dimethylformamide, and then adding hydroxylamine hydrochloride to continue the reaction to obtain the compound 17; and
enabling the compound 12 to react with 3-aminopyrazole in a mixed environment of N,N-diisopropylethylamine, N,N-dimethylformamide, and 2-(7-azabenzotriazolyl)-N,N,N′,N′-tetramethyluronium hexafluorophosphate to obtain compound 18.
